(19) United States
(12) Patent Application Publication
(10) Pub. No.: US 2018/0032699 A1
(43) Pub. Date: Feb. 1, 2018

(54) SYSTEM AND METHOD FOR MONITORING PHARMACEUTICAL USAGE

(71) Applicant: Thinkify LLC, Morgan Hill, CA (US)

(72) Inventors: Curtis L. Carrender, Morgan Hill, CA (US); Daniel Morrisey, San Jose, CA (US); Soneer Sainion, Morgan Hill, CA (US)

(21) Appl. No.: 15/662,834

(22) Filed: Jul. 28, 2017

Related U.S. Application Data
(60) Provisional application No. 62/367,817, filed on Jul. 28, 2016.

(51) Int. Cl. G06F 19/00; G06K 19/077; A61B 5/00

(52) U.S. Cl. CPC G06F 19/3456; A61B 5/4833; G06F 19/3462; G06K 19/07749; G06Q 50/22

(57) ABSTRACT

A pharmaceutical management system utilizing RFID implemented technology to monitor administration of pharmaceuticals and location of containers, the system designed for use with at least one container having an RFID tag associated with the respective container, the system including a monitoring apparatus having at least one position for placement of the container, a holder, a visual indicator associated with the holder, a reader associated with the holder and capable of wireless communication with the RFID tag on the container, and a remote management system that communicates with the reader in the monitoring apparatus and with authorized users to provide access to one or more of patient identification, pharmaceutical identification, dosage time, presence of the container on the holder, location of the container on the holder, and identification of authorized users who can access the information.

### BACKGROUND

### Technical Field

The present disclosure is directed to tracking the movement and administration of pharmaceuticals and, more particularly, to a radio-frequency identification (RFID) implementation of a remote monitoring station that enables users to determine whether one or more pharmaceuticals have been administered at the prescribed time.

### Description of the Related Art

It is often difficult to determine which pharmaceuticals or medications a patient has taken and when a particular medication is needed to be taken again. This is a concern for patients in a managed care situation because numerous patients need various medications at varying times throughout the day. Determining the proper time to take medications is also a particular concern for patients doing self-medication in the home situation. We are at a particular point in time where people are living longer, taking more medication, and also attempting to live more independent lifestyles. In some cases, those who need several medications throughout the day may also be less capable of monitoring their own pharmaceuticals, which includes the correct dosage and the period between medications. Often in the past, the caregiver for aging or infirm individuals was a family member. Today many people who would have previously been available to fill this caretaker role are working outside the home.

### BRIEF SUMMARY

The foregoing changes in our society have also coincided with improvements in technology that have heretofore been overlooked in improving the ability of individuals to manage their own health care. The first of these two is the ease of wireless connectivity with data being shared on the world wide web. The second of these is RFID (Radio Frequency Identification), allowing unique and inexpensive identification of individual items.

By combining all of these existing tools, along with a unique approach to notification, the present disclosure provides a system that allows management of pharmaceutical use, both easing the management of this task with the individual in the home setting and allowing management and monitoring of this task remotely.

In accordance with one aspect of the present disclosure, a pharmaceutical use monitoring system for use with at least one container containing a pharmaceutical and with an RFID tag associated with the respective at least one container is provided. The system includes a monitoring apparatus having a holder with a top surface, at least one position for placement of the container on the top surface, an indicator located at the at least one position, and a reader located on or in the holder and capable of wireless communication with the RFID tag on the at least one container; and a remote management system having a communication device capable of communicating electronically with the reader in the monitoring apparatus and of communicating with authorized users, the remote management system configured to receive and store information pertaining to the pharmaceutical in each of the at least one containers, the information including presence of the at least one container on the holder, and at least one from among a patient identification, pharmaceutical identification, pharmaceutical dosage time, location of the at least one container on the holder, and identification of authorized users who can access the information pertaining to the pharmaceutical in each of the at least one containers, the communication device in the remote management system further capable of communicating to authorized users and to the monitoring system at least one from among (a) a time to administer a dosage of the pharmaceutical from the respective at least one container, (b) a location of a container on the holder, and (c) a missing container.

In accordance with another aspect of the present disclosure, a method of monitoring a pharmaceutical using an RFID enabled pharmaceutical use monitoring system is provided. The method includes reading an RFID tag on at least one container using an RFID reader to obtain information about a medication stored in the container, the information including at least one from among a patient identification, pharmaceutical medication identification, dosage time, presence of the container on a holder, location of the container on the holder, and identification of authorized users who can access the information pertaining to the pharmaceutical medication in each of the at least one containers; transmitting the information read from the RFID tag on the at least one container to a remote management system; and receiving and storing the information transmitted from the RFID reader in the remote management system, transmitting to authorized users, and allowing access by authorized users to the received and stored information.

In accordance with another aspect of the method, a further step includes providing a physical indication at an intended location of the container of one from a time for taking a dose of medication in the container and a missing container. The physical indication can be a visual notification, an aural notification, or both the visual and the aural notification.

In accordance with a further aspect of the present disclosure, the visual notification comprises illumination of a light with a color, change of color, or visual perception of movement of the light.

In accordance with yet another aspect of the present disclosure, the method includes providing a website accessible to authorized users that provides access to the received and stored information.

### BRIEF DESCRIPTION OF THE SEVERAL VIEWS OF THE DRAWINGS

The foregoing and other features and advantages of the present disclosure will be more readily appreciated as the same become better understood from the following detailed description when taken in conjunction with the accompanying drawings, wherein:

FIG. 1 is a pictorial illustration an RFID pharmaceutical monitoring system formed in accordance with one implementation of the present disclosure;

FIG. 2 is a pictorial representation of a pharmaceutical use monitoring apparatus with each of three container locations occupied by a different container in accordance with the implementation of FIG. 1;

FIG. 3 is the pictorial representation of the pharmaceutical use monitoring apparatus of FIG. 2 with each of three container locations occupied by a different container, illustrating that the container location need not match the original location;

FIG. 4 is the pictorial representation of FIG. 2 illustrating activation of a visual indicator with one of the containers of medication in accordance with the present disclosure;

FIG. 5 is a pictorial representation of the pharmaceutical use monitoring apparatus of FIG. 2 with a visual indication of a missing container;

FIG. 6 is a pictorial illustration of the pharmaceutical use monitoring apparatus located inside a cabinet;

FIGS. 7A-7B illustrate notifications on a user's smartphone according to one implementation of the present disclosure;

FIG. 8 illustrates a screen display of the information available in a web site page available to authorized users of the RFID pharmaceutical use information system after log-in in accordance with one implementation of the present disclosure; and

FIG. 9 is a flowchart illustrating one example process of the RFID pharmaceutical use information system in accordance with one implementation of the present disclosure.

### DETAILED DESCRIPTION

In the following description, certain specific details are set forth in order to provide a thorough understanding of various implementations of the disclosure. For example, details about the construction and use of medication containers, bottles, cabinets, cell phones, and computers with associated monitors are not described. However, one skilled in the art will understand that the disclosure can be practiced without these specific details. In other instances, well-known manufacturing methods, structures, and processes associated with manufacturing techniques have not been described in detail to avoid unnecessarily obscuring the descriptions of the implementations of the present disclosure.

Unless the context requires otherwise, throughout the specification and claims that follow, the word “comprise” and variations thereof, such as “comprises” and “comprising,” are to be construed in an open, inclusive sense, that is, as “including, but not limited to.”

Reference throughout this specification to “one implementation” or “an implementation” means that a particular feature, structure or characteristic described in connection with the implementation is included in at least one implementation. Thus, the appearances of the phrases “in one implementation” or “in an implementation” in various places throughout this specification are not necessarily all referring to the same implementation. Furthermore, the particular features, structures, or characteristics can be combined in any suitable manner in one or more implementations.

As used in this specification and the appended claims, the singular forms “a,” “an,” and “the” include plural referents unless the context clearly dictates otherwise. It should also be noted that the term “or” is generally employed in its disjunctive sense.

As used in the specification and appended claims, the use of “correspond,” “corresponds,” and “corresponding” is intended to describe a ratio of or a similarity between referenced objects. The use of “correspond” or one of its forms should not be construed to mean the exact shape or size.

In the drawings, identical reference numbers identify similar elements or acts. The size and relative positions of elements in the drawings are not necessarily drawn to scale.

A brief overview of the system and its components is provided below followed by a detailed description of the system and its components in conjunction with the accompanying drawings. The word pharmaceutical is intended to have its ordinary meaning and to encompass and be equivalent to the word medication.

In one implementation of the present disclosure, an RFID tag is affixed to a medication container. The medication container is placed on a rack, shelf, stand, or other support that contains unique antennas for each area or location that a medication container or other pharmaceutical container might be placed. The RFID tag contains information about the associated container's contents and may contain information about the end user. It may also contain information about the recommended dosage of the pharmaceutical in the container and the interval between these dosages. The RFID reader's shelf, rack, stand, or other support also has visual indicators used for visually indicating the status of the dosage schedule the end user should follow. The RFID reader is connected to a web-based system that manages the use and dosage of each medication. A web-based tool is enabled to indicate to authorized users the status of the end user's medicinal history and prescription dosage schedule for the pharmaceutical.

Operation is as follows:

The RFID shelf, rack or table (identified herein as a support), reads a tag in location X. This information is then shared with the web-based information system managing the particular individual's pharmaceutical prescriptions and dosage. The web-based system links this particular RFID tag to a particular pharmaceutical or medication, or the RFID tag may already contain the information related to this particular medication or end user. The web-based management tool uses visual indicators on the RFID support to show the end user the status of their use for this medication. This information is also shared with authorized users of the system to indicate the status of the original patient's use of these pharmaceuticals. The web-based system is also used to notify authorized users of the status of movement of these medication containers. Some examples are as follows:

1) Medication A should be taken every 12 hours, yet there has been no movement of medication container A for 16 hours. The web-based system creates a visual notification on a support device to indicate to the end user that the medication has not been moved within the indicated proper time frame. At the same time, the end user is notified through their smart phone that he or she should take medication A. Concurrently an authorized user, in this case a relative, is notified on their smart phone through the web-based system that their relative has not moved medication A and is not following the recommended dosage requirements.

2) An end user patient is under doctor's orders to take each of 4 medications every day. The end user is following these instructions. A second authorized user such as a doctor or the patient's relative is able to access the secure web site and confirm that all of these medications have been moved during the day, giving the second authorized user some assurance that the proper dosage of the medications has been taken by or given to the patient.

3) A particular medication has not been confirmed or read by the reader in the support or in a cabinet or other storage area for some period of time, which is adjustable by authorized individuals. The web site sends out a notification to authorized users on the web site and smart phone or other electronic device that the medication is not located in its preferred location.

There has thus been outlined, rather broadly, some of the features of the present disclosure in order that the detailed description thereof may be better understood, and in order that the present contribution to the art may be better appreciated.

There are additional features of the present disclosure that will be described hereinafter and that will form the subject matter of the claims appended hereto. In this respect, before explaining at least one implementation of the present disclosure, it is to be understood that the present disclosure is not limited in its application to the details of construction or to the arrangements of the components set forth in the following description or illustrated in the drawings. The present disclosure is capable of other implementations and of being practiced and carried out in various ways. Also, it is to be understood that the phraseology and terminology employed herein are for the purpose of the description and should not be regarded as limiting.

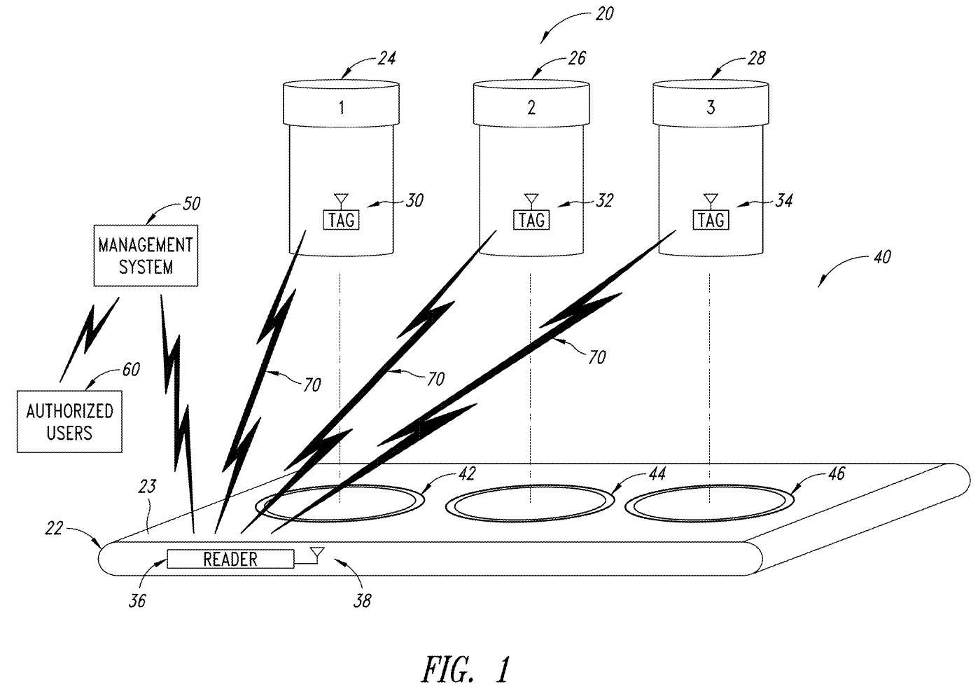

Referring initially to FIG. 1, shown therein is a pictorial representation of an RFID pharmaceutical use information system 20 formed in accordance with the present disclosure. The system 20 utilizes a monitoring apparatus that includes a holder 22, which can be a shelf, rack, table, or other form of support, on which are placed medication containers 24, 26, 28. Radio-frequency identifier (RFID) tags 30, 32, 34, which are affixed to or formed with respective medication containers 24, 26, 28, are used to track the movement of those items by being read by one or more RFID readers 36 or by any one of several antennas 38 from the same reader 36. Preferably the reader 36 is integrated into the holder 22, although it may be located remote from the holder 22 along with its antenna 38. Ideally the reader(s) 36 is located within the holder 22 with its antenna 38 to provide a self-contained monitoring apparatus 40. Such readers 36 are well-known and readily commercially available, and hence such will not be described in detail herein.

In one implementation, the holder 22 has a low-profile housing that is substantially flat on the top and bottom. This permits the holder 22 and the monitoring system 40 in general to be placed inside cupboards, cabinets, and other spaces with a fixed or non-adjustable height. This also facilitates storage of the monitoring system 40, such as in a briefcase or travel bag, in a manner similar to that of a laptop computer.

The monitoring apparatus 40 also includes visual indicators, preferably lights, such as LED lights 42, 44, 46, located on the holder 22 at each resting location of the respective container 24, 26, 28. Preferably the resting location is on the top surface 23 of the holder 22. Other visual indicators known in the art can also be used, such as neon lights, incandescent lights, and other well-known and commercially available electronic indicators. The light can be a simple small platform elevated or projecting above a top surface 23 of the holder 22, or a ring that is flush with a top surface of the holder 22. The light thus functions as the resting location for the respective container 24, 26, 28. Aural indicators may also be used in addition to or in place of the visual indicators, which would be of benefit to the visually impaired. Aural indicators can be a speaker built in to the housing of the holder 22 or other device that emits an audible sound and can likewise function as the resting location for the respective container 24, 26, 28.

The holder includes a self-contained rechargeable or replaceable battery source for electrical power. This facilitates portability of the monitoring apparatus 40. Alternatively, the source of electrical power for the holder 22, including one or more indicators and the reader 36, can be a plug-in source of power, such as a wall outlet.

The system 20 further includes a remote management system 50 that is remote to the monitoring apparatus 40 and that includes a communication device, such as a transceiver, that communicates via a wireless radio frequency communication network with the reader 36 via the reader's antenna 38. The remote management system 50 may also be directly wired to the reader 36 if desired. Various communication configurations may be used to provide for data transfer and control communications between the reader and the remote management system 50, which are known in the art and will not be described in detail herein. Such systems are described generally at the end of this detailed description. The management system 50 may be a dedicated server, computer, processor, or other known computing device having a transceiver (transmitter-receiver) capable of radio frequency communication or hard-wired communication, such as cable communication, and further capable of processing commands and instructions provided by software stored in a memory in the management system 50 or communicated to it. The management system 50 is also capable of transmitting information to remote users via the transceiver or hard-wired communication device, which preferably is via a secure protocol and of receiving commands and queries from remote users as described in more detail below.

Authorized users 60 may use their own personal devices, including without limitation cell phones, tablets, and personal computers, all of which are well known in the art, to communicate electronically, such as with wireless communication, with the management system 50 as will be described in more detail herein below. These authorized individuals, such as the user, their relatives or authorized friends and caregivers can be given access to use this RFID pharmaceutical tracking system to determine if a particular medication has been moved in a particular time period. For example, RFID tags may be on a set dosage time period, and if the medication container has not been moved within that time period, the visual indicator, in this case the respective LED 42, 44, 46 located at or surrounding the location of this medication container can change color and begin rotating to alert the end user. By “rotating” is meant the lighted ring is selectively illuminated in sections around the ring in a sequential manner such that the light appears to be traveling around the ring in a clockwise or counterclockwise manner.

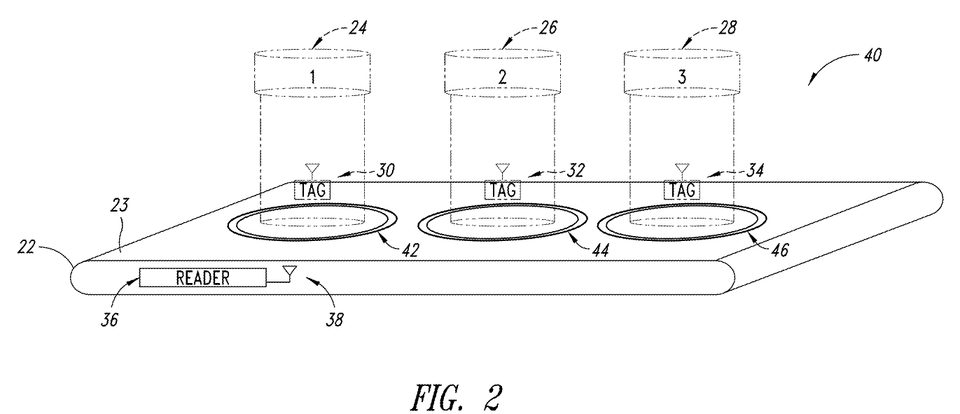

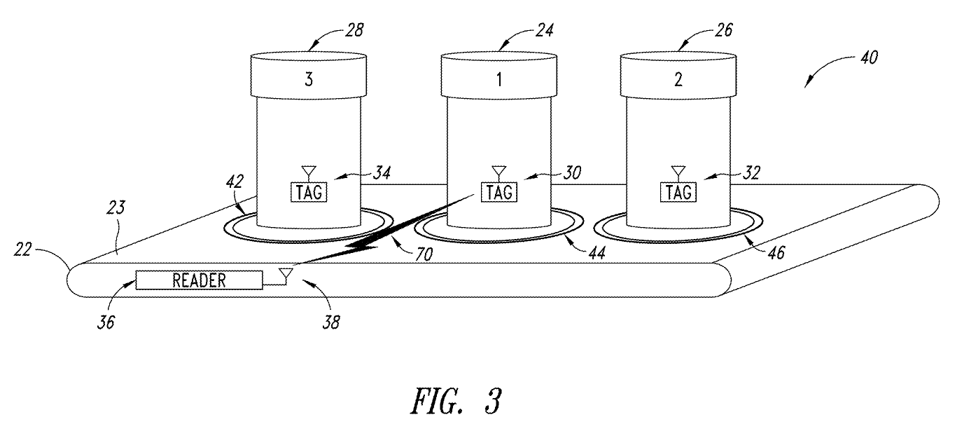

FIG. 2 shows the monitoring apparatus 40 with the medication containers 24, 26, 28 numbered 1 through 3 in phantom in place on the holder 22. The containers 24, 26, 28 may have any number of positions where they can be placed on the holder 22 as shown in FIG. 3. The RFID system needs only recognize a particular medication inside or on the pharmaceutical use information system. The containers 24, 26, 28 can be placed in any order so long as each container is in a unique location. The Figures illustrate three positions to simplify the description. However, the holder 22 can be configured or structured to accommodate more or less containers as desired.

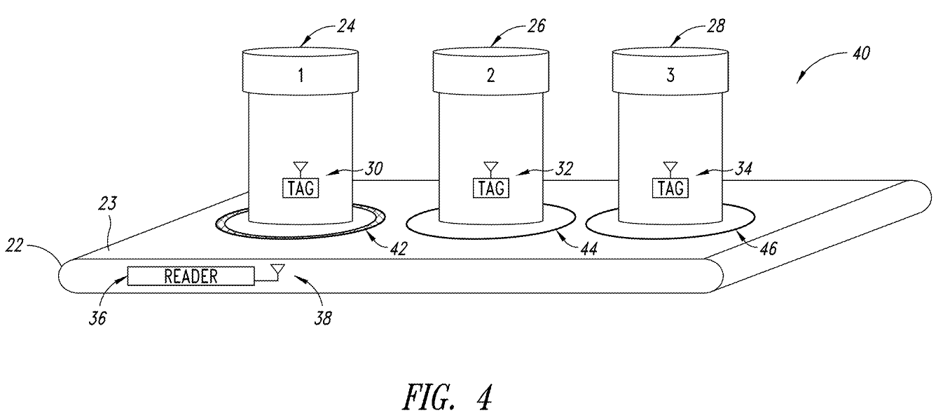

Based on signals 70 (shown in FIG. 3) received by the RFID reader 36 from within the holder 22, the system 20 may determine whether a container 24, 26, 28 has been moved based on the location of the corresponding tag 30, 32, 34 in the environment. In one implementation, the RFID reader 36 may look for a known inventory of pharmaceuticals on the holder 22 and indicate, based on algorithms related to the individual user involved, their prescription, the time and dosage, or that a particular item needs to be taken, or any combination of the foregoing. This is shown in FIG. 4 in which the ring of light 42 is illuminated (shown as cross-hatching). The lighted ring 42 provides a visual indication to the individual user that it is time to take the medication found in the container 24 at that location. This information is also communicated to the remote management system 50 and can be further communicated to authorized users 60 as shown in FIG. 1.

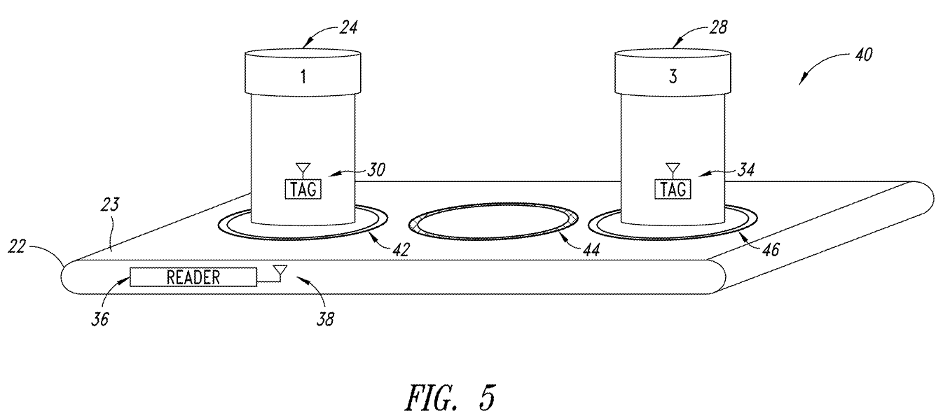

FIG. 5 shows an implementation where a particular inventory (container 26) has been taken or removed from the support. This container 26 was not seen by the reader when it was expected to be seen based upon rules set up in the reader 36 and or in the cloud based management system 50. In other words, the reader 36 transmits a signal to the tags 30, 32, 34 and only return signals are received from the tag 30 on the first container 24 and the tag 34 on the third container 28. No return signal was received from the tag 32 on the second container 26. The absence of the return signal from the tag 32 on the second container 26 is detected. In response to the detection of the absence of the return signal, the remote management system 50 tracks the time frame within which the container 26 has been moved. When the container 26 has not been moved within a required time frame or has been missing outside the time frame, the system 50 initiates a notification protocol in which the monitoring apparatus 40 indicates this situation with illumination of the indicator 44 on the pharmaceutical holder 22, and this information may also be communicated via the reader 36 and antenna 38 to the remote management system 50, and ultimately to the authorized user 60.

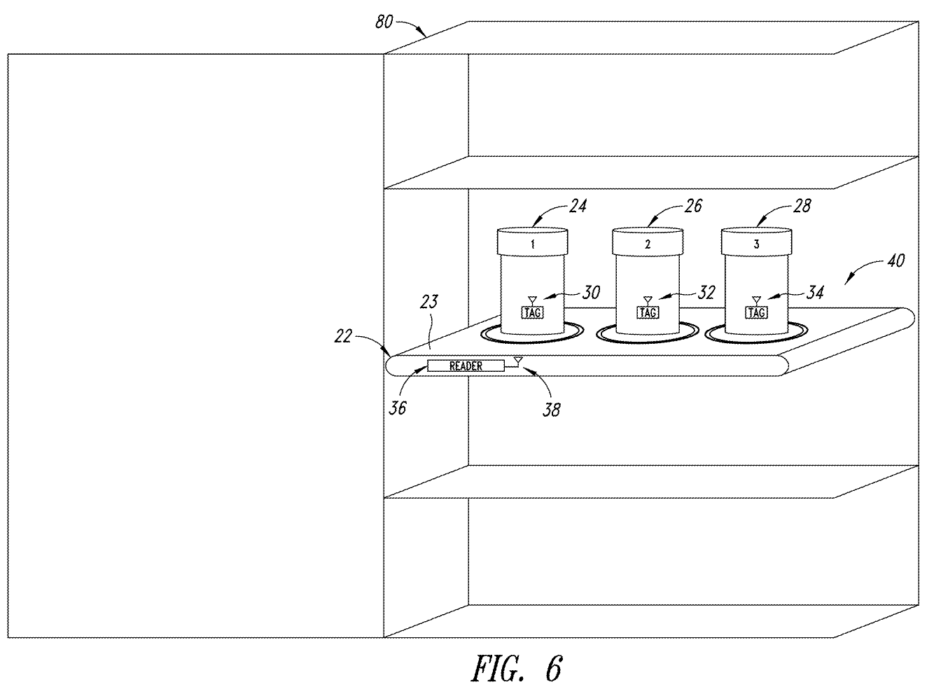

FIG. 6 shows that this particular approach is not limited to counter top applications, but can be placed inside a medication cabinet 80.

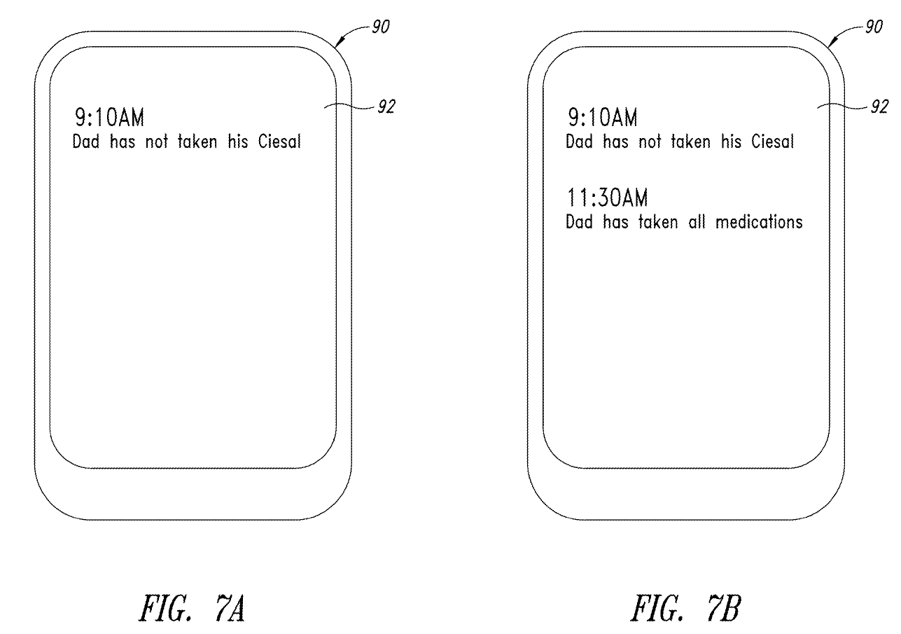

FIGS. 7A-7B show an example of a cell phone 90 receiving notifications on a display screen 92 about a particular pharmaceutical use information system and a particular user. In FIG. 7A the end user has not yet taken his medication. In the FIG. 7B case, this was finalized a short time later. Relatives, medical personnel or caregivers who are authorized by the end user can easily check up on pharmaceutical use by a third party. The third party, who may not be comfortable with management of all of their medical calendar needs, can easily take the correct medication at the correct times.

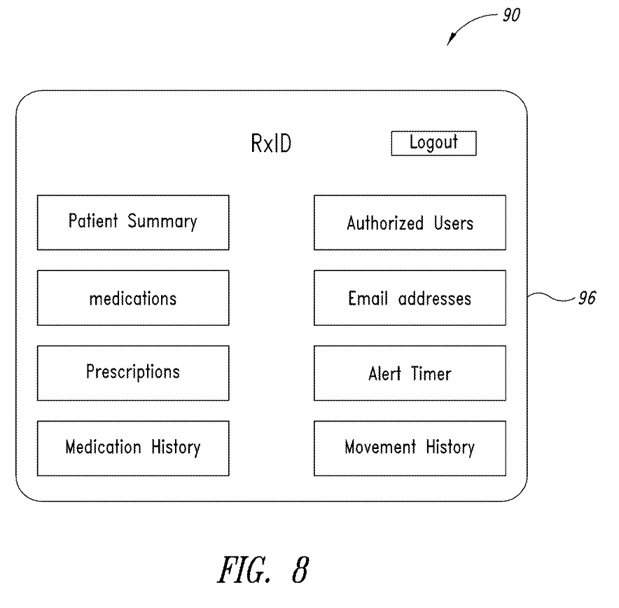

FIG. 8 shows an example of the web-based nature of the pharmaceutical use information system available after login to authorized users of the system 20. Here, a computer screen 96 displays a menu of tabs from which an authorized user can select using a touch screen or mouse. These features and abilities of the system and others may be available to third potential users once authorization is given, such as by logging in with a user name and password, as is conventional with most systems. Other security protocols may be used, which are well known to those skilled in the art.

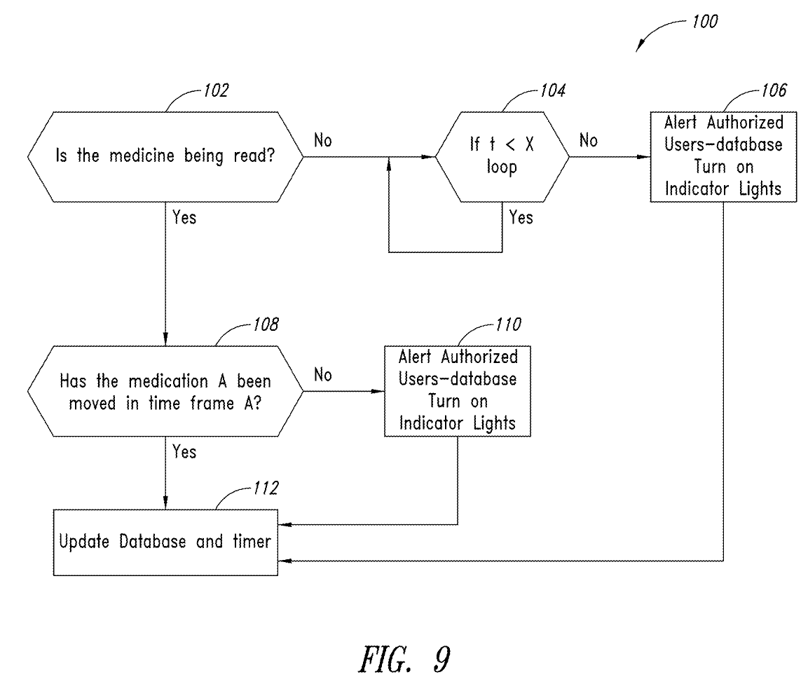

FIG. 9 shows a flow chart 100, which is one example of the logic that can be used to notify and set indicators on the system. This can be locally or remotely done. In the first box 102 the system checks to see if the medicine container is being read. If not, it checks a timer 104. Once the timer reaches a set value X the system alerts an authorized user's database in step 106 and turns on the indicator light for the appropriate medication container. If on the other hand the medication container is being read in step 102, then the system checks to see if the container has been moved within a predetermined time period in step 108. If not, the system alerts the user in step 110 and turns on the indicator light for the medication container. If on the other hand the container has been moved within the set time period, the database and the timer are reset or updated in step 112.

It is to be understood from the foregoing description that the monitoring apparatus 40 is controlled by an internal controller that determines the frequency at which the reader 36 polls the tags 30, 32, 36. In addition the controller responds to the received information from the reader 36 by sending signals to the indicators as described above. Thus, in an alternative implementation, the monitoring apparatus 40 would operate autonomous from the remote management system 50 and without the need for connection to a local or global network. In this implementation, users would not have access to the information obtained by the reader 36 from the tags 30, 32, 36.

As described herein, all measurements and determinations associated with one particular pharmaceutical item may also be performed across multiple items, locations, consumers, and time periods. In addition to being able to determine objective movement for a particular pharmaceutical item, the system may also determine timing information for the user to store for future reference. For example, the system may learn that someone normally takes medication A at 9:00 AM every day. While the timer may not require a notification until later in the day, the fact that the user did not take the medication at the regular time may enable a notification inside the normal notification window. Thus, the monitoring apparatus 40 independently or in conjunction with the system 50 is configured or programmed to track movement of a particular container and to determine a pattern of movement from detection signals sent from the reader 38. A threshold is set for a certain number of repeated detections at regular intervals, such as hours, days, weeks, or a combination of hours, days, and weeks. After the threshold number has been reached, the system sets a notification event for the detected intervals.

Relative movement may be an indicator of taking medication but there is no assurance of this. The medication may be simply moved for a moment from one location to another. As such there are time based controls in affect that determine if the medication was moved and how long it was in a position where the tag affixed to the medication was not read. If this time exceeds the time determined to be a minimum for taking that particular medication, the monitoring apparatus 40 or the management system 50 will initiate the notification protocol described herein for the medication not being taken.

The overall system 20 will also be useful in determining when a prescription needs to be filled. If for example it is known that 50 pills are in a particular prescription container where one pill is to be taken per day, and it is known that three days are needed between ordering a refill and actually receiving the new medication, then the system alerts the user or an authorized individual or both in response to detecting only 4 pills left in the container, which would correspond to 46 movements of that particular prescription (assuming each movement resulted in only one pill being taken).

The system 20 can be used to alert in response to other detections or indications not related to medication dosage. One example might be that the system has medication such as narcotics that are heavily regulated and often stolen or abused. If the system 20 sees that these particular medications are missing from the location at a particular time, it would alert an authorized user of the system, such as a physician, law enforcement, relative, pharmacist, or the like. In addition or in the alternative, the authorized end user would be given a tag that would allow use of the system without alert for any medication. In other words, the reader could recognize the end user through a personalized RFID tag that was not part of a pharmaceutical container.

The cloud based data site associated with this system could also be used to give indications to the end user of other items of interest. For example, because the system knows what medications are specific to this end user, the web site may also want to advertise or offer specific medications, vitamins, health aids, etc., that would also be useful to someone in this age bracket, medication group, or disease category.

The pharmaceutical use information system is connected by Wi-Fi, Bluetooth or other wireless or wired protocol to a network. This network is connected to the Internet. The system is also enabled to send text messages, Tweets, email or other forms of communication to any or all authorized users. All information, both current and historical, is available on the web page to authorized users.

The steps, software, data structures, and code described in this detailed description are typically stored on a computer readable storage medium, which may be any device or medium that can store code and data for use by a computer system. This includes, but is not limited to, magnetic and optical storage devices such as disk drives, magnetic tape, CDs (compact discs), DVDs (digital video discs), and computer instruction signals embodied in a transmission medium (with or without a carrier wave upon which the signals are modulated). For example, the transmission medium may include a telecommunications network, such as the Internet.

The present disclosure may be utilized upon any telecommunications network capable of transmitting data including voice data and other types of electronic data. Examples of suitable telecommunications networks for the present disclosure include but are not limited to global computer networks (e.g., Internet), wireless networks, cellular networks, satellite communications networks, cable communication networks (via a cable modem), microwave communications network, local area networks (LAN), wide area networks (WAN), campus area networks (CAN), metropolitan-area networks (MAN), and home area networks (HAN).

The present disclosure may communicate via a single telecommunications network or multiple telecommunications networks concurrently. Various protocols may be utilized by the electronic devices for communications such as but not limited to HTTP, SMTP, FTP and WAP (wireless Application Protocol). The present disclosure may be implemented upon various wireless networks such as but not limited to 3G, 4G, LTE, CDPD, CDMA, GSM, PDC, PHS, TDMA, FLEX, REFLEX, IDEN, TETRA, DECT, DATATAC, and MOBITEX. The present disclosure may also be utilized with online services and Internet service providers. The Internet is an exemplary telecommunications network for the present disclosure. The Internet is comprised of a global computer network having a plurality of computer systems around the world that are in communication with one another. Via the Internet, the computer systems are able to transmit various types of data between one another. The communications between the computer systems may be accomplished via various methods such as but not limited to wireless, Ethernet, cable, direct connection, telephone lines, and satellite.

The central communication unit may be comprised of any central communication site where communications are preferably established. The central communication units may be comprised of a server computer, cloud based computer, virtual computer, home computer or other computer system capable of receiving and transmitting data via IP networks and the telecommunication networks. As can be appreciated, a modem or other communication device may be required between each of the central communication units and the corresponding telecommunication networks. The central communication unit may be comprised of any electronic system capable of receiving and transmitting information (e.g., voice data, computer data, etc.).

The present disclosure is described above with reference to block and flow diagrams of systems, methods, apparatuses, and computer flow diagrams according to example implementations of the disclosure. It will be understood that one or more blocks of the block diagrams and flow diagrams, and combinations of blocks in the block diagrams and flow diagrams, respectively, can be implemented by computer executable program instructions. Likewise, some blocks of the block diagrams and flow diagrams may not necessarily need to be performed in the order presented, or may not necessarily need to be performed at all, according to some implementations of the disclosure. These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, implementations of the disclosure may provide for a computer program product, comprising a computer usable medium having a computer-readable program code or program instructions embodied therein, said computer-readable program code adapted to be executed to implement one or more functions specified in the flow diagram block or blocks.

The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks. Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

The present disclosure may be embodied in other specific forms without departing from the spirit or essential attributes thereof, and it is therefore desired that the present implementation be considered in all respects as illustrative and not restrictive. Many modifications and other implementations of the disclosure will come to mind to one skilled in the art to which this disclosure pertains and having the benefit of the teachings presented in the foregoing description and the associated drawings. Therefore, it is to be understood that the disclosure is not to be limited to the specific implementations disclosed and that modifications and other implementations are intended to be included within the scope of the appended claims. Although methods and materials similar to or equivalent to those described herein can be used in the practice or testing of the present disclosure, suitable methods and materials are described above. Thus, the present disclosure is not intended to be limited to the implementations shown, but is to be accorded the widest scope consistent with the principles and features disclosed herein.

The various implementations described above can be combined to provide further implementations. These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A pharmaceutical use monitoring system for use with at least one container containing a pharmaceutical and with an RFID tag associated with the respective at least one container, the system comprising:
a monitoring apparatus having a holder with a top surface, at least one position for placement of the container on the top surface, an indicator located at the at least one position, and a reader located on or in the holder and capable of wireless communication with the RFID tag on the at least one container; and
a remote management system having a communication device capable of communicating electronically with the reader in the monitoring apparatus and of communicating with authorized users, the remote management system configured to receive and store information pertaining to the pharmaceutical in each of the at least one containers, the information including presence of the at least one container on the holder, and at least one from among a patient identification, pharmaceutical identification, pharmaceutical dosage time, location of the at least one container on the holder, and identification of authorized users who can access the information pertaining to the pharmaceutical in each of the at least one containers, the communication device in the remote management system further capable of communicating to the authorized users and to the monitoring system at least one from among (a) a time to administer a dosage of the pharmaceutical from the respective at least one container, (b) a location of a container on the holder, and (c) a missing container.

2. The system of claim 1 wherein the indicator comprises a light.

3. A pharmaceutical use monitoring system, comprising:
at least one authorized user device capable of electronic communication;
at least one container having an RFID tag associated with the respective at least one container;
a monitoring apparatus having a top surface, at least one position for placement of the container on the top surface, a holder having an indicator located at the at least one position, and an RFID reader located on or in the holder and capable of wireless communication with the RFID tag on the at least one container; and
a remote management system having a communication device capable of communicating electronically with the reader in the monitoring apparatus and of communicating with authorized users, the remote management system configured to receive and store information pertaining to the pharmaceutical in each of the at least one containers, the information including presence of the at least one container on the holder, and at least one from among a patient identification, pharmaceutical identification, pharmaceutical dosage time, location of the at least one container on the holder, and identification of authorized users who can access the information pertaining to the pharmaceutical in each of the at least one containers, the communication device in the remote management system further capable of communicating to authorized users and to the monitoring system at least one from among (a) a time to administer a dosage of the pharmaceutical from the respective at least one container, (b) a location of a container on the holder, and (c) a missing container.

4. The system of claim 1 wherein the indicator is one or more of a visual indicator and an aural indicator.

5. A method of monitoring a pharmaceutical using an RFID enabled pharmaceutical use monitoring system, the method comprising:
reading an RFID tag on at least one container using an RFID reader to obtain information pertaining to a pharmaceutical stored in the at least one container, the information including presence of the at least one container on a holder, and at least one from among a patient identification, pharmaceutical identification, pharmaceutical dosage time, location of the at least one container on the holder, and identification of authorized users who can access the information pertaining to the pharmaceutical in each of the at least one containers;
transmitting to a remote management system the information read from the RFID tag on the at least one container;
receiving and storing the information transmitted from the RFID reader in the remote management system; and
transmitting the stored information to the authorized users, and allowing access by the authorized users to the transmitted stored information.

6. The method of claim 5, further comprising providing a physical indication at an intended location of the at least one container of one from a time for taking a dose of pharmaceutical in the container and a missing container.

7. The method of claim 6 wherein the physical indication comprises one of a visual notification, an aural notification, or both the visual and the aural notification.

8. The method of claim 7 wherein the visual notification comprises illumination of a light with a color, change of color, or visual perception of movement of the light.

9. The method of claim 7, comprising providing a website accessible to the authorized users that provides access to the received and stored information.
